(19) United States
(12) Patent Application Publication
(10) Pub. No.: US 2026/0029263 A1
(43) Pub. Date: Jan. 29, 2026

(54) BLOOD FLOW MEASUREMENT DEVICE

(71) Applicant: FUJIFILM Corporation, Tokyo (JP)

(72) Inventors: Yukito SAITOH, Minamiashigara-shi (JP); Makoto KAMO, Minamiashigara-shi (JP)

(73) Assignee: FUJIFILM Corporation, Tokyo (JP)

(21) Appl. No.: 19/181,375

(22) Filed: Apr. 17, 2025

Related U.S. Application Data
(63) Continuation of application No. PCT/JP2023/037635, filed on Oct. 18, 2023.

(30) Foreign Application Priority Data
Oct. 20, 2022 (JP) 2022-168286

(51) Int. Cl. G01F 1/661; A61B 5/026; G01F 1/66

(52) U.S. Cl. CPC G01F 1/661; A61B 5/0261; G01F 1/662

(57) ABSTRACT

An object of the present invention is to provide a thin blood flow measurement device. The object is accomplished by providing a blood flow measurement device that measures a blood flow by irradiating an object with near-infrared rays and measuring scattered light by the object, in which the blood flow measurement device includes at least one of a first configuration including an irradiation unit light guide plate that guides near-infrared rays emitted from a light source and an irradiation diffraction element that emits the near-infrared rays from the irradiation unit light guide plate, and a second configuration including a light receiving section light guide plate that guides the scattered light and a light receiving diffraction element that causes the scattered light by the object to be incident on the light receiving section light guide plate.

### CROSS-REFERENCE TO RELATED APPLICATIONS

This application is a Continuation of PCT International Application No. PCT/JP2023/037635 filed on Oct. 18, 2023, which claims priority under 35 U.S.C. § 119(a) to Japanese Patent Application No. 2022-168286 filed on Oct. 20, 2022. The above applications are hereby expressly incorporated by reference, in their entirety, into the present application.

### BACKGROUND OF THE INVENTION

### 1. Field of the Invention

The present invention relates to a blood flow measurement device that measures a blood flow of the head or the like.

### 2. Description of the Related Art

Measurement of blood flow rates in the brain, the muscle, the organ, and the like, and use of the measurement results for diagnosis of a physical function, health management, information mediation between a human body and a device, and the like are performed.

For example, with regard to the brain, a change in blood flow rate on a brain surface is detected, and the detected data are processed by a data processing device to acquire information indicating an activity state of the brain. In the blood flow measurement on the brain surface, a cerebral blood flow rate measurement device called a headset, which has a near-infrared irradiation unit and a near-infrared detection unit, is worn on a human head (forehead), the brain surface is irradiated with near-infrared rays, and the near-infrared rays scattered on the brain surface are received and measured, whereby a blood flow rate is measured.

As an example, JP2020-54649A describes a blood flow rate measurement device including a first main body part, a second main body part, and a hinge, in which the first main body part has a first housing including a first bottom surface, a light source that irradiates a portion other than the first housing from the first bottom surface with near-infrared rays, and a first light receiving portion that receives near-infrared rays from the first bottom surface side outside the first housing, the second main body part has a second housing including a second bottom surface and a second light receiving portion that receives near-infrared rays from the second bottom surface side outside the second housing, and the hinge connects the first main body part and the second main body part by allowing an angle formed between the first bottom surface and the second bottom surface to vary.

### SUMMARY OF THE INVENTION

It is desired to reduce the thickness of such a blood flow measurement device due to a demand for portability and the like.

However, in the blood flow measurement device, it is difficult to reduce the thickness since it is necessary to dispose the light source and the light receiving section directly above a measurement site.

In addition, a configuration in which the light source and the light receiving section are spaced from the measurement site by interposing an optical fiber is also considered. However, with this method, the optical system that emits the measurement light from the optical fiber to the measurement site and the optical system that introduces the diffused light from the measurement site into the optical fiber are large, making it difficult to reduce the thickness.

An object of the present invention is to solve such problems of the related art and to provide a blood flow measurement device which can be made thinner.

In order to accomplish the object, the present invention has the following configurations.

[1] A blood flow measurement device having:
an irradiation unit that has a light source emitting near-infrared rays and irradiates an object with the near-infrared rays; and
a light receiving section that has a light receiving element for measuring scattered light that is irradiated from the irradiation unit and scattered by the object,
in which in a case where a configuration in which the irradiation unit has an irradiation unit light guide plate that guides the near-infrared rays emitted from the light source and an irradiation diffraction element for emitting the near-infrared rays guided in the irradiation unit light guide plate from the irradiation unit light guide plate to irradiate the object with the near-infrared rays is defined as a first configuration, and
a configuration in which the light receiving section has a light receiving section light guide plate that guides the scattered light scattered by the object and a light receiving diffraction element for allowing the scattered light scattered by the object to be incident on the light receiving section light guide plate is defined as a second configuration,
the blood flow measurement device has at least one of the first configuration or the second configuration.

[2] The blood flow measurement device according to [1],
in which the blood flow measurement device has the first configuration and the second configuration.

[3] The blood flow measurement device according to [1] or [2],
in which at least one of the irradiation diffraction element or the light receiving diffraction element is a liquid crystal diffraction element.

[4] The blood flow measurement device according to [3],
in which the liquid crystal diffraction element includes a liquid crystal layer having a liquid crystal alignment pattern in which an orientation of an optical axis derived from the liquid crystal compound changes while continuously rotating along at least one in-plane direction.

[5] The blood flow measurement device according to [4],
in which a liquid crystal compound constituting the liquid crystal layer is helically twisted and aligned in a thickness direction.

[6] The blood flow measurement device according to [4],
in which the liquid crystal layer is a cholesteric liquid crystal layer.

[7] The blood flow measurement device according to [5],
in which the blood flow measurement device has two of the liquid crystal layers in which helical twisted directions of the liquid crystal compounds are different from each other.

[8] The blood flow measurement device according to [6],
in which the blood flow measurement device has two of the liquid crystal layers in which helical twisted directions of the liquid crystal compounds are different from each other.

[9] The blood flow measurement device according to any one of [1] to [8],
in which the blood flow measurement device has at least one of the first configuration having an incidence diffraction element for allowing the near-infrared rays emitted from the light source to be incident on the irradiation unit light guide plate, or
the second configuration having an emission diffraction element for emitting the scattered light guided in the light receiving section light guide plate from the light receiving section light guide plate to be incident on the light receiving element.

[10] The blood flow measurement device according to any one of [2] to [9],
in which the irradiation unit light guide plate and the light receiving section light guide plate are laminated.

[11] The blood flow measurement device according to any one of [2] to [9],
in which the irradiation unit light guide plate and the light receiving section light guide plate are arranged in a plane direction.

[12] The blood flow measurement device according to any one of [1] to [11],
in which at least one of the irradiation unit light guide plate or the light receiving section light guide plate has a first material and a second material having a refractive index higher than a refractive index of the first material, and
the second material is configured to be encompassed in the first material.

According to the present invention, the blood flow measurement device can be made thinner.

### BRIEF DESCRIPTION OF THE DRAWINGS

FIG. 1 is a view conceptually showing an example of a blood flow measurement device of an embodiment of the present invention.

FIG. 2 is a view conceptually showing an example of a liquid crystal diffraction element.

FIG. 3 is a plan view conceptually showing a liquid crystal layer of the liquid crystal diffraction element shown in FIG. 2.

FIG. 4 is a conceptual view showing an action of the liquid crystal layer shown in FIG. 3.

FIG. 5 is a conceptual view of an example of an exposure device that exposes an alignment film.

FIG. 6 is a conceptual view showing an action of the liquid crystal diffraction element.

FIG. 7 is a view conceptually showing another example of the liquid crystal diffraction element.

FIG. 8 is a plan view conceptually showing a liquid crystal layer of the liquid crystal diffraction element shown in FIG. 7.

FIG. 9 is a conceptual view showing an action of the liquid crystal diffraction element shown in FIG. 7.

FIG. 10 is a conceptual view showing an action of the liquid crystal diffraction element shown in FIG. 7.

FIG. 11 is a view conceptually showing another example of the blood flow measurement device of the embodiment of the present invention.

FIG. 12 is a view conceptually showing another example of the blood flow measurement device of the embodiment of the present invention.

### DESCRIPTION OF THE PREFERRED EMBODIMENTS

Hereinafter, a blood flow measurement device of an embodiment of the present invention will be described in detail based on suitable Examples shown in the accompanying drawings.

In the present specification, numerical ranges represented by “to” include numerical values before and after “to” as lower limit values and upper limit values.

In the present specification, “(meth)acrylate” is used to mean “either or both of acrylate and methacrylate”.

In the present specification, the meaning of “the same” includes a case where an error range is generally allowed in the technical field. In addition, in the present specification, the meaning of “all”, “entire”, or “entire surface” includes not only 100% but also a case in which an error range is generally allowable in the technical field, for example, 99% or more, 95% or more, or 90% or more.

In the present specification, near-infrared rays refer to light in a wavelength range of 650 to 1,400 nm.

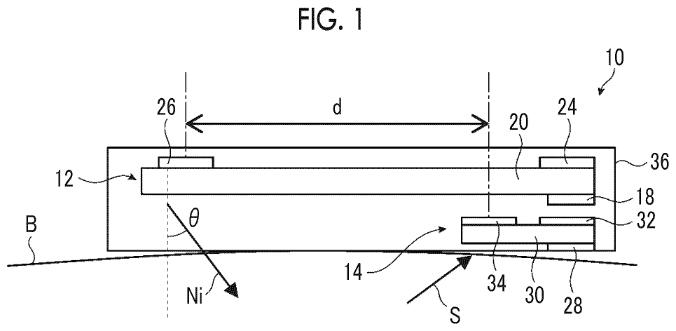

FIG. 1 conceptually shows an example of the blood flow measurement device of the embodiment of the present invention.

Furthermore, FIG. 1 is a view conceptually showing the blood flow measurement device of the embodiment of the present invention in order to describe the blood flow measurement device of the embodiment of the present invention. Accordingly, the size, the thickness, the positional relationship, the shape, and the like of each member are different from the actual ones. The same applies to other drawings.

A blood flow measurement device 10 shown in FIG. 1 has an irradiation unit 12 and a light receiving section 14.

The irradiation unit 12 has a light source unit 18, an irradiation unit light guide plate 20, an incidence diffraction element 24, and an irradiation diffraction element 26.

On the other hand, the light receiving section 14 has a light receiving element 28, a light receiving section light guide plate 30, an emission diffraction element 32, and a light receiving diffraction element 34.

Furthermore, the blood flow measurement device of the embodiment of the present invention is a device that measures a blood flow of an object B basically in the same manner as a known blood flow measurement device, except that the irradiation unit and/or the light receiving section has a light guide plate. Accordingly, various known methods can be used as a method for measuring a blood flow using near-infrared rays.

In the blood flow measurement device 10 in the example shown in the drawing, the irradiation unit 12 and the light receiving section 14 are accommodated in a case 36. In this way, the blood flow measurement device 10 prevents an object B or the like from coming into contact with the irradiation unit 12 and the light receiving section 14.

Furthermore, various known materials can be used as the case 36 as long as they do not come into contact with the irradiation unit 12 and the light receiving section 14 due to pressing or the like and have a strength to protect the irradiation unit 12 and the light receiving section 14.

The irradiation unit 12 and the light receiving section 14 are held (fixed) inside the case 36 in a laminated state without coming into contact with each other by a known method (not shown).

The light source unit 18 has a light source that emits near-infrared rays.

In the blood flow measurement device 10 of the embodiment of the present invention, the light source is not limited and a known light source can be used as long as it can emit the above-described near-infrared rays. Examples thereof include light emitting diodes (LED), laser diodes (LD), and vertical cavity surface emitting lasers (VCSEL).

The wavelength of the near-infrared rays emitted from the light source unit 18 is not limited and may be near-infrared rays in the above-described wavelength range.

Here, as described later, in the measurement of a blood flow using near-infrared rays, preferably, two types of near-infrared rays having different wavelengths are irradiated, the near-infrared rays scattered in the vicinity of, for example, the cerebral cortex of the object are measured, and the blood flow is measured from, for example, a ratio of the two types of received near-infrared rays.

Accordingly, it is preferable that the light source unit 18 emits two types of near-infrared rays having different wavelengths. Examples of the near-infrared rays include near-infrared rays having wavelengths of 730 nm and 810 nm, near-infrared rays having wavelengths of 730 nm and 855 nm, and near-infrared rays having wavelengths of 695 nm and 830 nm. In addition, from the viewpoint of improving the accuracy of the blood flow measurement, it is also preferable that the light source unit 18 emits three types of near-infrared rays having wavelengths of 735 nm, 810 nm, and 850 nm as near-infrared rays.

The near-infrared rays emitted from the light source may be polarized light or unpolarized light.

Here, in a case where the diffraction elements provided in the irradiation unit 12 and the light receiving section 14 are the liquid crystal diffraction elements which will be described later, the circularly polarized light is diffracted with high diffraction efficiency. Therefore, it is preferable that the near-infrared rays emitted from the light source unit 18 are circularly polarized light.

In this case, in a case where the near-infrared rays emitted from the light source are linearly polarized light, it is preferable that the light source unit 18 has a phase difference plate (¼ wave plate) for converting the emitted near-infrared rays into circularly polarized light. In addition, in a case where the near-infrared rays emitted from the light source are unpolarized light, it is preferable that the light source unit 18 has a linear polarizer and a phase difference plate for converting the near-infrared rays to be emitted into circularly polarized light.

In the blood flow measurement device 10, the irradiation unit 12 has an irradiation unit light guide plate 20 and the light receiving section 14 has a light receiving section light guide plate 30.

In the blood flow measurement device 10 of the embodiment of the present invention, the thinning of the blood flow measurement device is realized by using the light guide plate for the irradiation of the near-infrared rays in the irradiation unit 12 and receiving the scattered light from the object in the light receiving section 14.

Here, in the blood flow measurement device 10 in the example shown in the drawing, as a preferred aspect, both the irradiation unit 12 and the light receiving section 14 have a light guide plate. However, the present invention is not limited to this, and the light guide plate may be provided in at least one of the irradiation unit or the light receiving section.

Accordingly, the blood flow measurement device of the embodiment of the present invention may have a configuration in which the irradiation unit has only the light source unit without including the light guide plate, or may have a configuration in which the light receiving section has only the light receiving element without having the light guide plate, similarly to the known blood flow measurement device shown in JP2020-54649A and the like.

However, from the viewpoint that, for example, the blood flow measurement device can be more suitably thinned, it is preferable that both the irradiation unit and the light receiving section have a light guide plate as in the example shown in the drawing.

In the blood flow measurement device 10 of the embodiment of the present invention, the light guide plate is not limited and various well-known light guide plates used for a backlight unit of AR glasses and a liquid crystal display, or the like can be used.

In addition, a light guide plate having a so-called core-clad structure, in which a material having a high refractive index is encompassed in a material having a low refractive index, can also be used as the light guide plate. By using the light guide plate having a core-clad structure, near-infrared rays (scattered light) can be bent in the middle, and it is thus possible to make the blood flow measurement device thinner and more compact, and also improving the degree of freedom for miniaturization.

In the blood flow measurement device 10 in the example shown in the drawing, the light receiving section 14 has a light receiving element 28.

The light receiving element 28 receives and measures the scattered light with which the object B is irradiated by the irradiation unit 12 and scattered in the vicinity of, for example, the cerebral cortex of the object B.

The light receiving element 28 is not limited and various known light receiving elements (photoelectric conversion elements) can be used as long as they can measure the above-described near-infrared rays. Examples of the light receiving element include image sensors such as a photodiode, a phototransistor, and a complementary metal oxide semiconductor (CMOS) sensor.

In addition, the light receiving section 14 may have, as necessary, an amplifier that amplifies the signal emitted from the light receiving element 28, an analog digital (AD) converter that converts an analog signal emitted from the light receiving element 28 to a digital signal, and the like, in addition to the members shown in the drawing.

In the irradiation unit 12, the incidence diffraction element 24 and the irradiation diffraction element 26 are disposed in the vicinity of both end parts of the irradiation unit light guide plate 20. The light source unit 18 is disposed such that near-infrared rays are transmitted through the irradiation unit light guide plate 20 in the thickness direction and are incident on the incidence diffraction element 24.

On the other hand, in the light receiving section 14, the light receiving diffraction element 34 and the emission diffraction element 32 are disposed in the vicinity of both end parts of the light receiving section light guide plate 30. The above-described light receiving element 28 is disposed to receive the scattered light (near-infrared rays) diffracted by the emission diffraction element 32 and emitted from the light receiving section light guide plate 30.

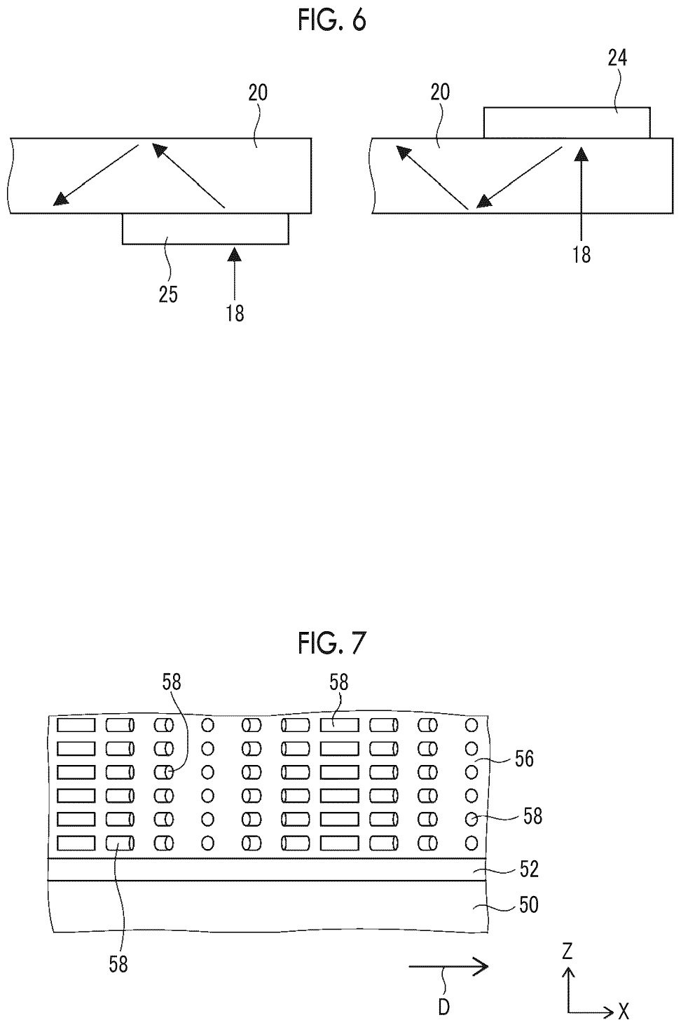

In the blood flow measurement device 10, the near-infrared rays emitted from the light source unit 18 are transmitted through the irradiation unit light guide plate 20, are incident on the incidence diffraction element 24, are diffracted by the incidence diffraction element 24 to be reflected, and are incident on the irradiation unit light guide plate 20 at an angle at which total reflection can occur (refer to the right side of FIG. 6).

The near-infrared rays incident on the irradiation unit light guide plate 20 are guided in the irradiation unit light guide plate 20 by repeating total reflection, and are incident on the irradiation diffraction element 26.

The near-infrared rays incident on the irradiation diffraction element 26 are diffracted by the irradiation diffraction element 26 to be reflected, and emitted toward the object B as indicated by an arrow Ni.

The near-infrared rays with which the object B is irradiated are scattered in the vicinity of, for example, the cerebral cortex of the object B. A part of the scattered light is emitted from the object B, is transmitted through the light receiving section light guide plate 30, and is incident on the light receiving diffraction element 34 as indicated by an arrow S.

The scattered light (near-infrared rays) incident on the light receiving diffraction element 34 is diffracted by the light receiving diffraction element 34 to be reflected, and a part of the light is incident on the light receiving section light guide plate 30 at an angle where total reflection can occur.

The scattered light incident on the light receiving section light guide plate 30 at an angle where total reflection can occur is guided in the light receiving section light guide plate 30 by repeating total reflection, and is incident on the emission diffraction element 32.

The near-infrared rays incident on the emission diffraction element 32 are diffracted by the irradiation diffraction element 26 to be reflected, are emitted from the light receiving section light guide plate 30, and are incident on the light receiving element 28 to be measured.

Accordingly, in the blood flow measurement device 10 of the embodiment of the present invention, it is preferable that the irradiation diffraction element 26 diffracts and irradiates near-infrared rays toward the light receiving diffraction element 34.

Here, in the blood flow measurement device 10 of the embodiment of the present invention, as conceptually shown in FIG. 1, in a case where a distance between the irradiation diffraction element 26 and the light receiving diffraction element 34 is represented by d [mm] and an angle formed between an irradiation direction of the near-infrared rays Ni from the irradiation diffraction element 26 and a normal direction of the irradiation diffraction element 26 is represented by θ [°],

it is preferable to satisfy:

θ/d>0.5.

Furthermore, the normal direction is a direction orthogonal to a surface of the object, and the normal direction of the irradiation diffraction element 26 is a direction orthogonal to the main surface of the irradiation diffraction element 26. The main surface is each of the maximum surfaces of a sheet-like material (a film, a plate-like material, a layer, or a film), that is, both surfaces in the thickness direction.

The distance d between the irradiation diffraction element 26 and the light receiving diffraction element 34 is not limited and may be appropriately set depending on a measurement site or the like. Here, the distance d is preferably more than 5 mm and 60 mm or less (5<d≤60 [mm]). The distance d is more preferably 10 to 50 mm.

Furthermore, the distance d is a distance in the plane direction between the irradiation diffraction element 26 and the light receiving diffraction element 34, which does not include the lamination direction, as shown in FIG. 1. In other words, the distance d is a distance between the irradiation diffraction element 26 and the light receiving diffraction element 34 on a plane in a case where the light guide plate is viewed in the normal direction.

By satisfying the conditions, the scattered light of the near-infrared rays scattered by the cerebral cortex of the brain, the arm blood vessel, and the like are more suitably received by the light receiving section 14 by irradiating the object B, and the measurement of the blood flow can be accurately performed.

Furthermore, the distance d between the irradiation diffraction element 26 and the light receiving diffraction element 34 is a distance between the centers of the centers of the both. The center of the diffraction element is a normal center according to the planar shape of the diffraction element.

For example, in a case where the diffraction element is circular, the center of the diffraction element is the center of the circle, and in a case where the diffraction element is rectangular, the center of the diffraction element is the intersection of the diagonals. In a case where the shape of the diffraction element is another shape, a circle inscribed in the diffraction element may be assumed and the center of the circle may be the center of the diffraction element. Furthermore, in the present invention, the rectangle also includes a square.

In the blood flow measurement device 10, the incidence diffraction element 24 of the irradiation unit 12 and the emission diffraction element 32 of the light receiving section 14 are provided as preferred aspects.

Accordingly, in the blood flow measurement device of the embodiment of the present invention, the incidence diffraction element 24 may not be provided, and the light source unit 18 may allow near-infrared rays to be incident on the irradiation unit guide plate 20 at an angle at which total reflection can occur from the edge face of the irradiation unit guide plate 20. In addition, the blood flow measurement device of the embodiment of the present invention may not include the emission diffraction element 32 and the light receiving element 28 may receive the scattered light emitted from the edge face of the light receiving section light guide plate 30.

However, by using the diffraction element for incidence and emission of light into and from the light guide plate, the thickness of the blood flow measurement device can be more suitably reduced. Accordingly, the blood flow measurement device of the embodiment of the present invention preferably includes at least one of the incidence diffraction element 24 of the irradiation unit 12 or the emission diffraction element 32 of the light receiving section 14, and more preferably includes the both.

In the blood flow measurement device 10 in the example shown in the drawing, the incidence diffraction element 24 and the irradiation diffraction element 26 of the irradiation unit 12, and the emission diffraction element 32 and the light receiving diffraction element 34 of the light receiving section 14 are all reflection-type diffraction elements. However, the present invention is not limited thereto and these diffraction elements may be transmission-type diffraction elements.

In addition, the incidence diffraction element 24 and the irradiation diffraction element 26 of the irradiation unit 12, and the emission diffraction element 32 and the light receiving diffraction element 34 of the light receiving section 14 are typically diffraction elements of the same type, but may be a mixture of diffraction elements of different types.

The incidence diffraction element 24 and the irradiation diffraction element 26 of the irradiation unit 12, and the emission diffraction element 32 and the light receiving diffraction element 34 of the light receiving section 14 are not limited, and various well-known diffraction elements can be used. Examples of the diffraction element include a liquid crystal diffraction element, a surface relief diffraction element, and a hologram diffraction element.

Among these, the liquid crystal diffraction element is suitably used from the viewpoint that, for example, it is easily made thinner, and can provide a large diffraction angle and a high diffraction efficiency.

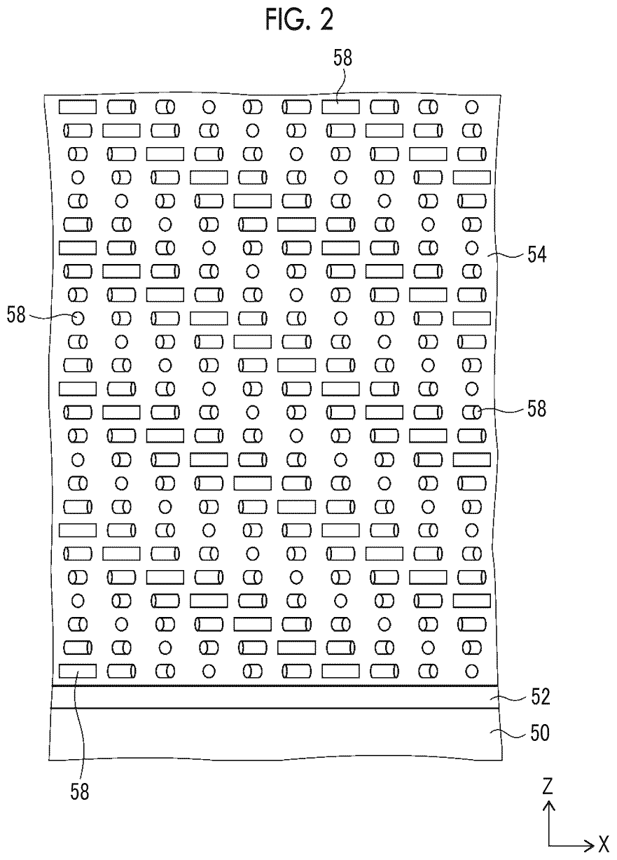

FIG. 2 conceptually shows an example of a reflection-type liquid crystal diffraction element.

As conceptually shown in FIG. 2, the liquid crystal diffraction element has a support 50, an alignment film 52, and a cholesteric liquid crystal layer 54 as a liquid crystal layer that exhibits an action as a diffraction element.

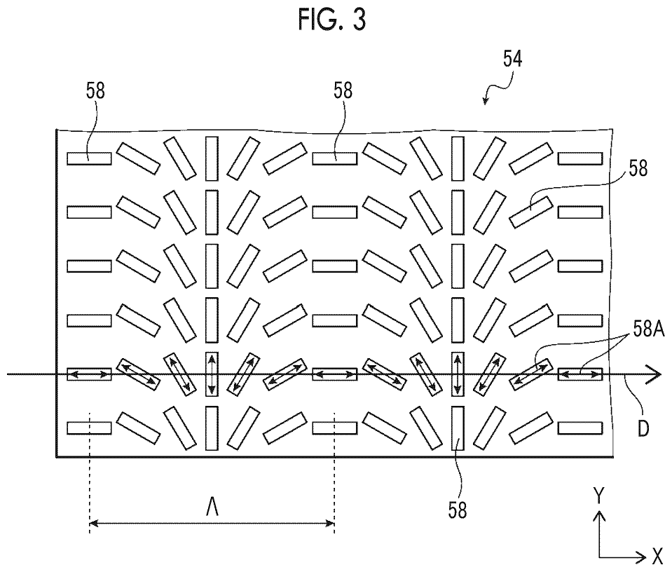

FIG. 3 conceptually shows the alignment state of a liquid crystal compound in a plane of the main surface of the cholesteric liquid crystal layer 54.

In the following description, it is assumed that the main surface of the cholesteric liquid crystal layer 54 is an X-Y plane and a cross section perpendicular to the X-Y plane is an X-Z plane. That is, FIG. 2 corresponds to a schematic view of the X-Z plane of the cholesteric liquid crystal layer 54, and FIG. 3 corresponds to a schematic view of the X-Y plane of the cholesteric liquid crystal layer 54.

As shown in FIG. 2, the cholesteric liquid crystal layer 54 is a layer obtained by cholesteric alignment of the liquid crystal compound. In addition, FIGS. 2 and 3 show an example in which the liquid crystal compound forming the cholesteric liquid crystal layer is a rod-like liquid crystal compound.

Furthermore, the liquid crystal diffraction element shown in FIG. 2 includes the support 50, the alignment film 52, and the cholesteric liquid crystal layer 54, but the present invention is not limited thereto. The liquid crystal diffraction element may have only the alignment film 52 and the cholesteric liquid crystal layer 54, obtained by, for example, bonding to a light guide plate (the irradiation unit light guide plate 20 and the light receiving section light guide plate 30), and then peeling the support 50. Alternatively, the liquid crystal diffraction element may have only the cholesteric liquid crystal layer 54 obtained by bonding to a light guide plate and then peeling the support 50 and the alignment film 52.

### <Support>

The support 50 supports the alignment film 52 and the cholesteric liquid crystal layer 54.

As the support 50, various sheet-like materials (films or plate-like materials) can be used as long as they can support the alignment film 52 and the cholesteric liquid crystal layer 54.

Furthermore, the transmittance of the support 50 to near-infrared rays is preferably 50% or more, more preferably 70% or more, and still more preferably 85% or more.

The thickness of the support 50 is not limited, and the thickness with which the alignment film 52 and the cholesteric liquid crystal layer 54 can be supported may be appropriately set depending on a material for forming the support 50, and the like.

The thickness of the support 50 is preferably 1 to 2,000 μm, more preferably 3 to 500 μm, and still more preferably 5 to 250 μm.

The support 50 may be a single layer or a multi-layer.

In a case where the support 50 is a single layer, examples thereof include a support 50 consisting of glass, triacetyl cellulose (TAC), polyethylene terephthalate (PET), a polycarbonate, polyvinyl chloride, acryl, a polyolefin, and the like. In a case where the support 50 is a multi-layer, examples thereof include a support that includes any of the above-described single-layered supports as a substrate, and has another layer provided on a surface of the substrate.

### <Alignment Film>

In the liquid crystal diffraction element, the alignment film 52 is formed on a surface of the support 50.

The alignment film 52 is an alignment film for aligning the liquid crystal compound 58 to a predetermined liquid crystal alignment pattern during the formation of the cholesteric liquid crystal layer 54.

Although described later, in the present invention, the cholesteric liquid crystal layer 54 has a liquid crystal alignment pattern in which an orientation of an optical axis 58A (refer to FIG. 3) derived from the liquid crystal compound 58 changes while continuously rotating along one in-plane direction. Accordingly, the alignment film 52 is formed such that the cholesteric liquid crystal layer 54 can form the liquid crystal alignment pattern.

In the following description, an expression, “the orientation of the optical axis 58A rotates” will also be simply referred to as an expression, “the optical axis 58A rotates”.

As the alignment film 52, various well-known films can be used.

Examples of the alignment film include a rubbed film formed of an organic compound such as a polymer, an obliquely deposited film formed of an inorganic compound, a film having a microgroove, and a film formed by lamination of Langmuir-Blodgett (LB) films formed with a Langmuir-Blodgett's method using an organic compound such as ω-tricosanoic acid, dioctadecylmethylammonium chloride, and methyl stearate.

The alignment film 52 formed by a rubbing treatment can be formed by rubbing a surface of a polymer layer with paper or fabric in a given direction multiple times.

As the material used for the alignment film 52, for example, a material for forming polyimide, polyvinyl alcohol, the polymer having a polymerizable group described in JP1997-152509A (JP-H9-152509A), or the alignment film 52 such as JP2005-97377A, JP2005-99228A, and JP2005-128503A is preferable.

In the liquid crystal diffraction element, for example, the alignment film 52 can be suitably used as a so-called photo-alignment film obtained by irradiating a photo-alignment material with polarized light or unpolarized light to obtain the alignment film 52. That is, in the liquid crystal diffraction element, a photo-alignment film formed by applying a photo-alignment material to the support 50 is suitably used as the alignment film 52.

The irradiation of polarized light can be performed in a direction orthogonal or oblique to the photo-alignment film, and the irradiation of unpolarized light can be performed in an oblique direction with respect to the photo-alignment film.

Preferable examples of the photo-alignment material used in the alignment film that can be used in the present invention include: an azo compound described in JP2006-285197A, JP2007-76839A, JP2007-138138A, JP2007-94071A, JP2007-121721A, JP2007-140465A, JP2007-156439A, JP2007-133184A, JP2009-109831A, JP3883848B, and JP4151746B; an aromatic ester compound described in JP2002-229039A; a maleimide- and/or alkenyl-substituted nadiimide compound having a photo-alignable unit described in JP2002-265541A and JP2002-317013A; a photocrosslinking silane derivative described in JP4205195B and JP4205198B, a photocrosslinking polyimide, a photocrosslinking polyamide, or a photocrosslinking polyester described in JP2003-520878A, JP2004-529220A, and JP4162850B; and a photodimerizable compound, in particular, a cinnamate compound, a chalcone compound, or a coumarin compound described in JP1997-118717A (JP-H9-118717A), JP1998-506420A (JP-H10-506420A), JP2003-505561A, WO2010/150748A, JP2013-177561A, and JP2014-12823A.

Among these, the azo compound, the photocrosslinking polyimide, the photocrosslinking polyamide, the photocrosslinking polyester, the cinnamate compound, or the chalcone compound is suitably used.

The thickness of the alignment film 52 is not limited and the thickness with which a required alignment function can be obtained may be appropriately set depending on the material for forming the alignment film 52.

The thickness of the alignment film 52 is preferably 0.01 to 5 μm, and more preferably 0.05 to 2 μm.

A method for forming the alignment film 52 is not limited and various well-known methods corresponding to the material for forming the alignment film 52 can be used. As an example, a method in which an alignment film 52 is applied onto a surface of the support 50 and dried, and then, the alignment film 52 is exposed to laser light to form an alignment pattern is exemplified.

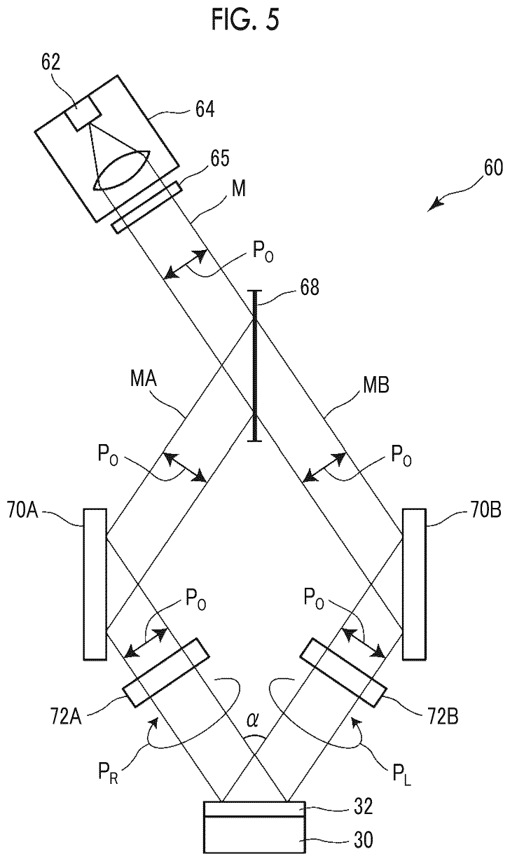

FIG. 5 conceptually shows an example of an exposure device that exposes the alignment film 52 to form an alignment pattern.

An exposure device 60 shown in FIG. 5, a light source 64 including a laser 62, a λ/2 plate 65 that changes a polarization direction of laser light M emitted from the laser 62, a polarized beam splitter 68 that splits the laser light M emitted from the laser 62 into two rays MA and MB, mirrors 70A and 70B that are disposed on optical paths of the split two rays MA and MB, and λ/4 plates 72A and 72B.

Furthermore, the light source 64 emits linearly polarized light P₀. The λ/4 plate 72A converts the linearly polarized light P₀ (rays MA) into dextrorotatory circularly polarized light P_{R}, and the λ/4 plate 72B converts the linearly polarized light P₀ (rays MB) into levorotatory circularly polarized light P_{L}.

The support 50 having the alignment film 52 on which the alignment pattern is not yet formed is disposed at an exposed portion, the two rays MA and MB intersect and interfere with each other on the alignment film 52, and the alignment film 52 is irradiated with and exposed to the interference light.

Due to the interference in this case, the polarization state of light with which the alignment film 52 is irradiated periodically changes according to interference fringes. This makes it possible to obtain an alignment film having the alignment pattern in which the alignment states periodically change. In the following description, this alignment film having the alignment pattern will also be referred to as “patterned alignment film”.

In the exposure device 60, the period of the alignment pattern can be adjusted by changing an intersection angle α between the two rays MA and MB. That is, by adjusting the intersecting angle α in the exposure device 60, in the alignment pattern in which the optical axis 58A derived from the liquid crystal compound 58 continuously rotates along the one in-plane direction, the length of the single period over which the optical axis 58A rotates by 180° in the one direction in which the optical axis 58A rotates can be adjusted.

By forming the cholesteric liquid crystal layer on the alignment film 52 having the alignment pattern in which such an alignment state periodically changes, as described later, the cholesteric liquid crystal layer 54 having the liquid crystal alignment pattern in which the optical axis 58A derived from the liquid crystal compound 58 continuously rotates along the one in-plane direction can be formed.

In addition, by rotating the optical axes of the λ/4 plates 72A and 72B by 90°, respectively, a rotation direction of the optical axis 58A can be reversed.

As described above, the patterned alignment film has an alignment pattern to obtain a liquid crystal alignment pattern in which the liquid crystal compound is aligned such that the orientation of the optical axis of the liquid crystal compound in the liquid crystal layer formed on the patterned alignment film changes while continuously rotating along at least one in-plane direction.

In a case where an axis directed along the orientation in which the liquid crystal compound is aligned is referred to as an alignment axis, it can be said that the patterned alignment film has an alignment pattern in which the orientation of the alignment axis changes while continuously rotating along at least one direction of in-plane directions. The alignment axis of the patterned alignment film can be detected by measuring absorption anisotropy. For example, in a case where the patterned alignment film is irradiated with linearly polarized light while rotating the patterned alignment film and the amount of light transmitted through the patterned alignment film is measured, the orientation with which the amount of light is maximum or minimum is observed by gradually changing along one in-plane direction.

Furthermore, in the present invention, the alignment film 52 is provided as a preferred aspect and is not a configuration requirement.

For example, the following configuration can also be adopted, in which by forming an alignment pattern on a support 50 using a method of subjecting the support 50 to a rubbing treatment, a method of processing the support 50 with laser light, or the like, the liquid crystal layer has a liquid crystal alignment pattern in which the orientation of the optical axis 58A derived from the liquid crystal compound 58 changes while continuously rotating along at least one in-plane direction. That is, in the present invention, the support 50 may be set to act as the alignment film.

### <Cholesteric Liquid Crystal Layer>

In the reflection-type liquid crystal diffraction element, the cholesteric liquid crystal layer 54 is formed on a surface of the alignment film 52.

The cholesteric liquid crystal layer 54 is a liquid crystal layer that is obtained by fixing a cholesteric liquid crystal phase and has a liquid crystal alignment pattern in which an orientation of an optical axis derived from a liquid crystal compound changes while continuously rotating along at least one in-plane direction.

As conceptually shown in FIG. 2, the cholesteric liquid crystal layer 54 has a helical structure in which the liquid crystal compound 58 is helically turned and laminated as in a cholesteric liquid crystal layer obtained by fixing a typical cholesteric liquid crystal phase. In the helical structure, a configuration in which the liquid crystal compound 58 is helically rotated once (rotated by 360°) and laminated is set as one helical pitch (helical pitch P), and plural pitches of the helically turned liquid crystal compound 58 are laminated.

As is well known, the cholesteric liquid crystal phase exhibits selective reflectivity at a specific wavelength.

The selective reflection center wavelength λ (center wavelength λ of the selective reflection) in the general cholesteric liquid crystal phase depends on a helical pitch P in the cholesteric liquid crystal phase, and is based on a relationship between an average refractive index n of the cholesteric liquid crystal phase and λ=n×P. Therefore, the selective reflection center wavelength can be adjusted by adjusting the helical pitch P. The selective reflection center wavelength of the cholesteric liquid crystal phase increases as the helical pitch P increases.

As described above, the blood flow measurement device of the embodiment of the present invention measures a blood flow of the object B by irradiating the object B with near-infrared rays. Accordingly, the helical pitch P of the cholesteric liquid crystal layer 54 is set depending on the wavelength of the near-infrared rays as the measurement light.

The helical pitch P of the cholesteric liquid crystal phase depends on a type of a chiral agent used together with the liquid crystal compound and an addition concentration of the chiral agent during the formation of the cholesteric liquid crystal layer. Accordingly, a desired helical pitch P can be obtained by adjusting these conditions.

Furthermore, the adjustment of the helical pitch P is described in detail in FUJIFILM Research Report No. 50 (2005), p. 60 to 63. As a method for measuring a sense of helix and a helical pitch P, a method described in “Introduction to Experimental Liquid Crystal Chemistry”, (the Japanese Liquid Crystal Society, 2007, Sigma Publishing Co., Ltd.), p. 46, and “Liquid Crystal Handbook” (the Editing Committee of Liquid Crystal Handbook, Maruzen Publishing Co., Ltd.), p. 196 can be used.

As is well known, the cholesteric liquid crystal phase exhibits selective reflectivity with respect to levorotatory or dextrorotatory circularly polarized light at a specific wavelength. Whether or not the reflected light is dextrorotatory circularly polarized light or levorotatory circularly polarized light is determined depending on a helical twisted direction (sense) of the cholesteric liquid crystal phase. With regard to the selective reflection of the circularly polarized light by the cholesteric liquid crystal phase, in a case where the helical twisted direction of the cholesteric liquid crystal phase is right, dextrorotatory circularly polarized light is reflected, and in a case where the helical twisted direction of the cholesteric liquid crystal phase is left, levorotatory circularly polarized light is reflected.

Furthermore, the revolution direction of the cholesteric liquid crystal phase can be adjusted by adjusting the type of the liquid crystal compound that forms the cholesteric liquid crystal layer and/or the kind of the chiral agent to be added.

In addition, a half-width Δλ (nm) of a selective reflection range (circularly polarized light reflection range) where selective reflection is exhibited depends on Δn of the cholesteric liquid crystal phase and the helical pitch P and complies with a relationship of Δλ=Δn×P. Therefore, the width of the selective reflection range can be controlled by adjusting Δn. Δn can be adjusted by adjusting a kind of a liquid crystal compound for forming the cholesteric liquid crystal layer and a mixing ratio thereof, and a temperature during alignment fixation.

The half-width of the reflection wavelength range is adjusted according to the use of the blood flow measurement device 10, and for example, the half-width may be 10 to 500 nm, and is preferably 20 to 300 nm, and more preferably 30 to 100 nm.

As shown in FIG. 3, in the X-Y plane of the cholesteric liquid crystal layer 54, the liquid crystal compounds 58 are arranged along a plurality of arrangement axes D parallel to the X-Y plane. On each of the arrangement axes D, the orientation of the optical axis 58A of the liquid crystal compound 58 changes while continuously rotating in the one in-plane direction along the arrangement axis D. Here, for the convenience of description, it is assumed that the arrangement axis D is directed to the X direction. In addition, in the Y direction, the liquid crystal compounds 58 in which the orientations of the optical axes 58A are the same are aligned at regular intervals.

Furthermore, “the orientation of the optical axis 58A of the liquid crystal compound 58 changes while continuously rotating in the one in-plane direction along the arrangement axis D” means that angles formed between the optical axes 58A of the liquid crystal compounds 58 and the arrangement axes D vary depending on positions in the arrangement axis D direction, and the angles formed between the optical axes 58A and the arrangement axes D gradually change from θ to θ+180° or θ−180° along the arrangement axis D. That is, in each of the plurality of liquid crystal compounds 58 arranged along the arrangement axis D, as shown in FIG. 3, the optical axis 58A changes along the arrangement axis D while rotating on a certain angle basis.

Furthermore, a difference between the angles of the optical axes 58A of the liquid crystal compounds 58 adjacent to each other in the arrangement axis D direction is preferably 45° or less, more preferably 15° or less, and still more preferably the less angles.

In addition, in the present specification, in a case where the liquid crystal compound 58 is a rod-like liquid crystal compound, the optical axis 58A of the liquid crystal compound 58 is intended to mean a molecular major axis of the rod-like liquid crystal compound. On the other hand, in a case where the liquid crystal compound 58 is a disk-like liquid crystal compound, the optical axis 58A of the liquid crystal compound 58 is intended to mean an axis parallel to the normal direction with respect to a disc plane of the disk-like liquid crystal compound.

In the cholesteric liquid crystal layer 54, in the liquid crystal alignment pattern of such a liquid crystal compound 58, the length (distance) over which the optical axis 58A of the liquid crystal compound 58 rotates by 180° in the arrangement axis D direction in which the optical axis 58A changes while continuously rotating in the plane is the length A of the single period in the liquid crystal alignment pattern.

That is, a distance between the centers of two liquid crystal compounds 58 in the arrangement axis D direction is the length A of the single period, the two liquid crystal compounds 58 having the same angle in the arrangement axis D direction. Specifically, as shown in FIG. 3, a distance between centers in the arrangement axis D direction of two liquid crystal compounds 58 in which the arrangement axis D direction and the direction of the optical axis 58A match each other is the length A of the single period. In the following description, the length A of the single period will also be referred to as a “single period A”.

In the liquid crystal alignment pattern of the cholesteric liquid crystal layer 54, the single period A is repeated in the arrangement axis D direction, that is, in the one in-plane direction in which the orientation of the optical axis 58A changes while continuously rotating. In the liquid crystal diffraction element, the single period A is the period of the diffraction structure.

On the other hand, in the liquid crystal compound 58 forming the cholesteric liquid crystal layer 54, the orientations of the optical axes 58A are the same in the direction (in FIG. 3, the Y direction) orthogonal to the arrangement axis D direction, that is, the Y direction orthogonal to one direction in which the optical axis 58A continuously rotates.

In other words, in the liquid crystal compound 58 forming the cholesteric liquid crystal layer 54, the angles formed between the optical axes 58A of the liquid crystal compound 58 and the arrangement axis D (X direction) are the same in the Y direction.

Hereinafter, an action of diffraction of the cholesteric liquid crystal layer will be described.

In a cholesteric liquid crystal layer of the related art, a helical axis derived from a cholesteric liquid crystal phase is perpendicular to the main surface (X-Y plane), and a reflecting surface thereof is parallel to the main surface (X-Y plane). In addition, the optical axis of the liquid crystal compound is not tilted with respect to the main surface (X-Y plane). In other words, the optical axis is parallel to the main surface (X-Y plane). Accordingly, in a case where the X-Z plane of the cholesteric liquid crystal layer in the related art is observed with an SEM, an arrangement direction in which bright portions and dark portions are alternately arranged is perpendicular to the main surface (X-Y plane).

The cholesteric liquid crystal phase has specular reflectivity, and therefore, in a case where light is incident from the normal direction into the cholesteric liquid crystal layer, the light is reflected in the normal direction.

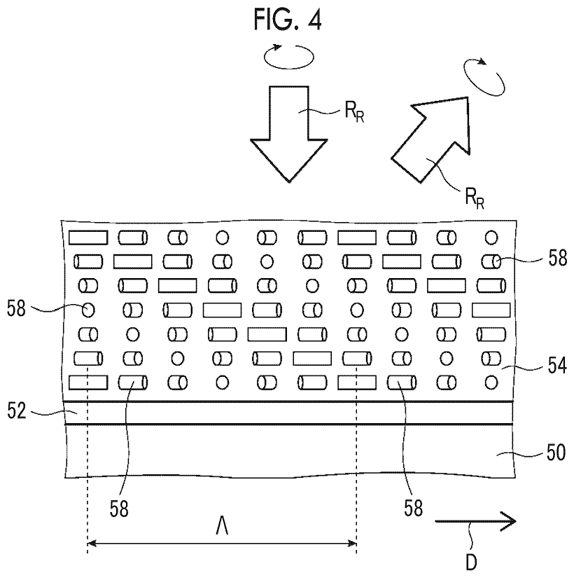

In contrast, the cholesteric liquid crystal layer 54 reflects incidence rays in a state where the light is tilted in the arrangement axis D direction with respect to specular reflection. The cholesteric liquid crystal layer 54 has the liquid crystal alignment pattern in which the optical axis 58A changes while continuously rotating along the arrangement axis D direction in the plane (the predetermined one in-plane direction). Hereinafter, the description will be made with reference to FIG. 4.

For example, the cholesteric liquid crystal layer 54 selectively reflects dextrorotatory circularly polarized light R_{R} of red light. Accordingly, in a case where light is incident on the cholesteric liquid crystal layer 54, the cholesteric liquid crystal layer 54 reflects only dextrorotatory circularly polarized light R_{R} of red light and allows transmission of the other light.

In the cholesteric liquid crystal layer 54, the optical axis 58A of the liquid crystal compound 58 changes while rotating along the arrangement axis D direction (the one in-plane direction).

In addition, the liquid crystal alignment pattern formed in the cholesteric liquid crystal layer 54 is a pattern that is periodic in the arrangement axis D direction. Therefore, as conceptually shown in FIG. 4, the dextrorotatory circularly polarized light R_{R} of red light incident on the cholesteric liquid crystal layer 54 is reflected (diffracted) in a direction corresponding to the period of the liquid crystal alignment pattern, and the reflected dextrorotatory circularly polarized light R_{R} of red light is reflected (diffracted) in a direction tilted with respect to the XY plane (the main surface of the cholesteric liquid crystal layer) in the arrangement axis D direction.

As a result, in a case where the cholesteric liquid crystal layer 54 is applied to a blood flow measurement device or the like, it can be used as a diffraction element in which light incident from a direction perpendicular to the main surface of the light guide plate can be reflected (diffracted) at an angle at which total reflection occurs in the light guide plate and the light guided in the light guide plate by total reflection can be reflected (diffracted) in a direction perpendicular to the main surface of the light guide plate.

In the cholesteric liquid crystal layer 54, by appropriately setting the direction of the arrangement axis D as the one in-plane direction in which the optical axis 58A rotates, the reflection direction (diffraction direction) of light can be adjusted.

In addition, in a case where circularly polarized light having the same wavelength and the same revolution direction is reflected, by reversing the rotation direction of the optical axis 58A of the liquid crystal compound 58 toward the arrangement axis D direction, a reflection direction of the circularly polarized light can be reversed.

That is, in FIGS. 2 and 3, the rotation direction of the optical axis 58A toward the arrangement axis D direction is clockwise, and one circularly polarized light is reflected in a state where it is tilted in the arrangement axis D direction. By setting the rotation direction of the optical axis 58A to be counterclockwise, the circularly polarized light is reflected in a state where it is tilted in a direction opposite to the arrangement axis D direction.

Further, in the liquid crystal layer having the same liquid crystal alignment pattern, the reflection direction is reversed by adjusting the helical revolution direction of the liquid crystal compound 58, that is, the revolution direction of circularly polarized light to be reflected.

For example, in a case where the helical revolution direction is right-twisted, the liquid crystal layer selectively reflects dextrorotatory circularly polarized light, and has the liquid crystal alignment pattern in which the optical axis 58A rotates clockwise along the arrangement axis D direction. As a result, the dextrorotatory circularly polarized light is reflected in a state where it is tilted in the arrangement axis D direction.

In addition, for example, in a case where the helical revolution direction is left-twisted, the liquid crystal layer selectively reflects levorotatory circularly polarized light, and has the liquid crystal alignment pattern in which the optical axis 58A rotates clockwise along the arrangement axis D direction. As a result, the levorotatory circularly polarized light is reflected in a state where it is tilted in a direction opposite to the arrangement axis D direction.

In the liquid crystal diffraction element, in the liquid crystal alignment pattern of the liquid crystal compound in the liquid crystal layer, the single period Λ as the length over which the optical axis of the liquid crystal compound rotates by 180° is the period (single period) of the diffraction structure. In addition, in the liquid crystal layer, the one in-plane direction (arrangement axis D direction) in which the optical axis of the liquid crystal compound changes while rotating is the periodic direction of the diffraction structure.

In the blood flow measurement device 10, the length of the single period Λ of the liquid crystal diffraction element is not limited.

Here, in the liquid crystal diffraction element (cholesteric liquid crystal layer) having the liquid crystal alignment pattern, as the single period Λ decreases, the angle of reflected light with respect to the incidence rays increases. That is, as the single period Λ decreases, reflected light can be reflected in a state where it is largely tilted with respect to incidence rays. For example, in a case where light is incident from the normal direction of the liquid crystal diffraction element, the angle formed between the traveling direction of the reflected light and the normal direction increases as the single period Λ decreases.

Accordingly, the length of the single period Λ of the liquid crystal diffraction element may be appropriately set depending on the incidence angle into the light guide plate, the magnitude of diffraction of light to be emitted from the light guide plate, and the like.

In addition, with regard to the single period Λ of the irradiation diffraction element 26, the length of the single period Λ is preferably set so that an angle θ [°] formed by the irradiation direction of the near-infrared rays Ni from the irradiation diffraction element 26 and the normal direction of the irradiation diffraction element 26 satisfies the above-described “θ/d>0.5” in accordance with a distance d [mm] between the irradiation diffraction element 26 and the light receiving diffraction element 34.

The length of the single period of the liquid crystal diffraction element is preferably about 0.3 to 1 times and more preferably about 0.4 to 0.9 times the wavelength of the near-infrared rays used as the measurement light. By setting the length of the single period of the liquid crystal diffraction element to be in this range, it is possible to suitably allow near-infrared rays to be incident on the light guide plate and to be totally reflected and guided.

### <<Method for Forming Cholesteric Liquid Crystal Layer>>

The cholesteric liquid crystal layer 54 can be formed by fixing a liquid crystal phase in which a liquid crystal compound is aligned in a predetermined alignment state in a layer shape. For example, the cholesteric liquid crystal layer can be formed by fixing a cholesteric liquid crystal phase in a layer shape.

The structure in which a cholesteric liquid crystal phase is fixed may be a structure in which the alignment of the liquid crystal compound as a liquid crystal phase is fixed. Typically, the structure in which a liquid crystal phase is fixed is preferably a structure which is obtained by making the polymerizable liquid crystal compound to be in a state where a predetermined liquid crystal phase is aligned, polymerizing and hardening the polymerizable liquid crystal compound with ultraviolet irradiation, heating, or the like to form a layer having no fluidity, and concurrently changing the state of the polymerizable liquid crystal compound into a state where the alignment state is not changed by an external field or an external force.

Furthermore, in the structure in which the liquid crystal phase is fixed, it is sufficient that the optical properties of the liquid crystal phase are maintained, and the liquid crystal compound 58 in the liquid crystal layer may not exhibit liquid crystallinity. For example, the polymerizable liquid crystal compound may be made to have a high molecular weight by a hardening reaction and therefore the liquid crystallinity may be lost.

Examples of a material used for forming the liquid crystal layer include a liquid crystal composition including a liquid crystal compound. It is preferable that the liquid crystal compound is a polymerizable liquid crystal compound.

In addition, the liquid crystal composition used for forming the liquid crystal layer may further include a surfactant and a chiral agent.

### ——Polymerizable Liquid Crystal Compound——

The polymerizable liquid crystal compound may be a rod-like liquid crystal compound or a disk-like liquid crystal compound.

Examples of the rod-like polymerizable liquid crystal compound include a rod-like nematic liquid crystal compound. As the rod-like nematic liquid crystal compound, an azomethine compound, an azoxy compound, a cyanobiphenyl compound, a cyanophenyl ester compound, a benzoate compound, a phenyl cyclohexanecarboxylate compound, a cyanophenylcyclohexane compound, a cyano-substituted phenylpyrimidine compound, an alkoxy-substituted phenylpyrimidine compound, a phenyldioxane compound, a tolane compound, or an alkenylcyclohexylbenzonitrile compound is preferably used. Not only a low-molecular-weight liquid crystal compound but also a high-molecular-weight liquid crystal compound can be used.

The polymerizable liquid crystal compound can be obtained by introducing a polymerizable group into the liquid crystal compound. Examples of the polymerizable group include an unsaturated polymerizable group, an epoxy group, and an aziridinyl group, and among these, the unsaturated polymerizable group is preferable, and the ethylenically unsaturated polymerizable group is more preferable. The polymerizable group can be introduced into a molecule of the liquid crystal compound by various methods. The number of the polymerizable groups in the polymerizable liquid crystal compound is preferably 1 to 6, and more preferably 1 to 3.

Examples of the polymerizable liquid crystal compound include the compounds described in Makromol. Chem., (1989), Vol. 190, p. 2255, Advanced Materials (1993), Vol. 5, p. 107, U.S. Pat. Nos. 4,683,327A, 5,622,648A, 5,770,107A, WO95/22586A, WO95/24455A, WO97/00600A, WO98/23580A, WO98/52905A, JP1989-272551A (JP-H1-272551A), JP1994-16616A (JP-H6-16616A), JP1995-110469A (JP-H7-110469A), JP1999-80081A (JP-H11-80081A), JP2001-328973A, and the like. Two or more kinds of the polymerizable liquid crystal compounds may be used in combination. In a case where two or more polymerizable liquid crystal compounds are used in combination, the alignment temperature can be lowered.

In addition, as a polymerizable liquid crystal compound other than the above-described examples, for example, such a cyclic organopolysiloxane compound having a cholesteric phase as described in JP1982-165480A (JP-S57-165480A) can be used. Further, as the above-described polymer liquid crystal compound, for example, a polymer in which a liquid crystal mesogenic group is introduced into the main chain, a side chain, or both the main chain and a side chain, a polymer cholesteric liquid crystal in which a cholesteryl group is introduced into a side chain, such a liquid crystal polymer as described in JP1997-133810A (JP-H9-133810A), and such a liquid crystal polymer as described in JP1999-293252A (JP-H11-293252A) can be used.

### ——Disk-Like Liquid Crystal Compound——

As the disk-like liquid crystal compound, for example, the compounds described in JP2007-108732A or JP2010-244038A can be preferably used.

In addition, the addition amount of the polymerizable liquid crystal compound in the liquid crystal composition is preferably 75% to 99.9% by mass, more preferably 80% to 99% by mass, and still more preferably 85% to 90% by mass with respect to the solid content mass (mass excluding a solvent) of the liquid crystal composition.

### ——Surfactant——

The liquid crystal composition used for forming the liquid crystal layer may contain a surfactant.

It is preferable that the surfactant is a compound that can function as an alignment control agent contributing to the stable or rapid alignment of a cholesteric liquid crystal phase. Examples of the surfactant include a silicone-based surfactant and a fluorine-based surfactant, and preferred examples thereof include the fluorine-based surfactant.

Specific examples of the surfactant include the compounds described in paragraphs [0082] to [0090] of JP2014-119605A, the compounds described in paragraphs [0031] to [1134] of JP2012-203237A, the compounds exemplified in paragraphs [0092] and [0093] of JP2005-99248A, the compounds exemplified in paragraphs [0076] to [0078] and paragraphs [0082] to [0085] of JP2002-129162A, and the fluorine (meth)acrylate polymers described in paragraphs [0018] to [0043] of JP2007-272185A, and the like.

Furthermore, the surfactants may be used alone or in combination of two or more kinds thereof.

As the fluorine-based surfactant, the compounds described in paragraphs to of JP2014-119605A are preferable.

The addition amount of the surfactant in the liquid crystal composition is preferably 0.01% to 10% by mass, more preferably 0.01% to 5% by mass, and still more preferably 0.02% to 1% by mass with respect to the total mass of the liquid crystal compound.

### ——Chiral Agent (Optically Active Compound)——

The chiral agent has a function of causing a helical structure of a cholesteric liquid crystal phase to be formed. The chiral agent may be selected depending on the purpose since a helical twisted direction or a helical pitch derived from the compound varies.

The chiral agent is not particularly limited, and a well-known compound (for example, Liquid Crystal Device Handbook (No. 142 Committee of Japan Society for the Promotion of Science, 1989), Chapter 3, Article 4-3, chiral agent for twisted nematic (TN) or super twisted nematic (STN), p. 199), isosorbide, an isomannide derivative, or the like can be used.

The chiral agent generally includes an asymmetric carbon atom, but an axially asymmetric compound or a planar asymmetric compound, including no asymmetric carbon atom, can also be used as the chiral agent. Examples of the axially asymmetric compound or the planar asymmetric compound include binaphthyl, helicene, paracyclophane, and derivatives thereof. The chiral agent may also have a polymerizable group. In a case where both the chiral agent and the liquid crystal compound have a polymerizable group, a polymer having a repeating unit induced from the polymerizable liquid crystal compound and a repeating unit induced from the chiral agent can be formed by a polymerization reaction between the polymerizable chiral agent and the polymerizable liquid crystal compound. In this aspect, it is preferable that the polymerizable group in the polymerizable chiral agent is the same as the polymerizable group in the polymerizable liquid crystal compound. Accordingly, the polymerizable group of the chiral agent is preferably an unsaturated polymerizable group, an epoxy group, or an aziridinyl group, more preferably an unsaturated polymerizable group, and still more preferably an ethylenically unsaturated polymerizable group.

In addition, the chiral agent may be a liquid crystal compound.

In a case where the chiral agent has a photoisomerization group, a pattern having a desired reflection wavelength corresponding to a luminescence wavelength can be formed by irradiation of an actinic ray or the like through a photo mask after coating and alignment, which is preferable. As the photoisomerization group, an isomerization site of a compound exhibiting photochromic properties, an azo group, an azoxy group, or a cinnamoyl group is preferable. As specific compounds, the compounds described in JP2002-80478A, JP2002-80851A, JP2002-179668A, JP2002-179669A, JP2002-179670A, JP2002-179681A, JP2002-179682A, JP2002-338575A, JP2002-338668A, JP2003-313189A, JP2003-313292A, or the like can be used.

The content of the chiral agent in the liquid crystal composition is preferably 0.01% to 200% by mole, and more preferably 1% to 30% by mole with respect to the molar content of the liquid crystal compound.

### ——Polymerization Initiator——

In a case where the liquid crystal composition includes a polymerizable compound, it is preferable that the liquid crystal composition contains a polymerization initiator. In an aspect where a polymerization reaction progresses with ultraviolet irradiation, it is preferable that the polymerization initiator is a photopolymerization initiator which initiates a polymerization reaction with ultraviolet irradiation.

Examples of the photopolymerization initiator include α-carbonyl compounds (described in the specifications of U.S. Pat. Nos. 2,367,661A and 2,367,670A), acyloin ether (described in the specification of U.S. Pat. No. 2,448,828A), α-hydrocarbon-substituted aromatic acyloin compounds (described in the specification of U.S. Pat. No. 2,722,512A), polynuclear quinone compounds (described in the specifications of U.S. Pat. Nos. 3,046,127A and 2,951,758A), combinations of triarylimidazole dimer and p-aminophenyl ketone (described in the specification of U.S. Pat. No. 3,549,367A), acridine compounds and phenazine compounds (described in JP1985-105667A (JP-S60-105667A) and the specification of U.S. Pat. No. 4,239,850A), and oxadiazole compounds (described in the specification of U.S. Pat. No. 4,212,970A).

A content of the photopolymerization initiator in the liquid crystal composition is preferably 0.1% to 20% by mass, and more preferably 0.5% to 12% by mass with respect to the content of the liquid crystal compound.

### ——Crosslinking Agent——

In order to improve the film hardness after hardening and to improve durability, the liquid crystal composition may optionally include a crosslinking agent. As the crosslinking agent, a crosslinking agent that cures the liquid crystal composition with ultraviolet rays, heat, humidity, and the like can be suitably used.

The crosslinking agent is not particularly limited and can be appropriately selected depending on the purpose. Examples of the crosslinking agent include a polyfunctional acrylate compound such as trimethylol propane tri(meth)acrylate and pentaerythritol tri(meth)acrylate; an epoxy compound such as glycidyl (meth)acrylate and ethylene glycol diglycidyl ether; an aziridine compound such as 2,2-bis hydroxymethyl butanol-tris [3-(1-aziridinyl)propionate] and 4,4-bis(ethyleneiminocarbonylamino)diphenylmethane; an isocyanate compound such as hexamethylene diisocyanate and a biuret type isocyanate; a polyoxazoline compound having an oxazoline group at a side chain thereof; and an alkoxysilane compound such as vinyl trimethoxysilane and N-(2-aminoethyl)-3-aminopropyltrimethoxysilane. In addition, a known catalyst can be used depending on reactivity of the crosslinking agent, and in addition to improving the film hardness and the durability, productivity can be improved. These may be used alone or in combination of two or more kinds thereof.

The content of the crosslinking agent is preferably 3% to 20% by mass, and more preferably 5% to 15% by mass with respect to the solid content mass of the liquid crystal composition. In a case where the content of the crosslinking agent is in the range, an effect of improving a crosslinking density can be easily obtained and the stability of a liquid crystal phase is further improved.

### ——Other Additives——

Optionally, a polymerization inhibitor, an antioxidant, an ultraviolet absorber, a light stabilizer, a coloring material, metal oxide fine particles, or the like can be added to the liquid crystal composition in a range where optical performance and the like do not deteriorate.

In a case where the cholesteric liquid crystal layer is formed, it is preferable that the liquid crystal composition is used as liquid.

The liquid crystal composition may include a solvent. The solvent is not limited and can be appropriately selected depending on the purpose, but an organic solvent is preferable.

The organic solvent is not limited and can be appropriately selected depending on the purpose. Examples of the organic solvent include ketones, alkyl halides, amides, sulfoxides, heterocyclic compounds, hydrocarbons, esters, and ethers. These may be used alone or in combination of two or more kinds thereof. Among these, the ketone is preferable in consideration of an environmental burden.

In a case where the cholesteric liquid crystal layer is formed, it is preferable that the liquid crystal layer is formed by applying the liquid crystal composition to a surface where the cholesteric liquid crystal layer is to be formed, aligning the liquid crystal compound to a state of a liquid crystal phase, and hardening the liquid crystal compound.

That is, in a case where the cholesteric liquid crystal layer is formed on the alignment film 52, it is preferable that the liquid crystal layer obtained by fixing a cholesteric liquid crystal phase is formed by applying the liquid crystal composition to the alignment film 52, aligning the liquid crystal compound to a state of a cholesteric liquid crystal phase, and hardening the liquid crystal compound.

For the application of the liquid crystal composition, a printing method such as ink jet or scroll printing or a well-known method such as spin coating, bar coating, or spray coating capable of uniformly applying liquid to a sheet-like material can be used.

The applied liquid crystal composition is optionally dried and/or heated and then cured to form the liquid crystal layer. In the drying and/or heating step, the liquid crystal compound in the liquid crystal composition may be aligned to a cholesteric liquid crystal phase. In the case of heating, the heating temperature is preferably 200° C. or lower, and more preferably 130° C. or lower.

The aligned liquid crystal compound is further polymerized as necessary. With regard to the polymerization, either of thermal polymerization and photopolymerization using light irradiation may be performed, but the photopolymerization is preferable. With regard to the light irradiation, ultraviolet ray is preferably used. The irradiation energy is preferably 20 mJ/cm² to 50 J/cm², and more preferably 50 to 1,500 mJ/cm². In order to promote a photopolymerization reaction, the light irradiation may be performed under heating conditions or in a nitrogen atmosphere. A wavelength of the ultraviolet rays to be emitted is preferably 250 to 430 nm.

The thickness of the cholesteric liquid crystal layer 54 is not limited, and the thickness with which a required light reflectivity can be obtained may be appropriately set depending on the use of the diffraction element, the light reflectivity required for the liquid crystal layer, the material for forming the cholesteric liquid crystal layer 54, and the like.

In the blood flow measurement device 10 of the embodiment of the present invention, the reflection-type liquid crystal diffraction element may include two cholesteric liquid crystal layers.

Specifically, the liquid crystal diffraction element preferably has two cholesteric liquid crystal layers, that is, a cholesteric liquid crystal layer that selectively reflects dextrorotatory circularly polarized light and a cholesteric liquid crystal layer that selectively reflects levorotatory circularly polarized light. With such a configuration, the near-infrared rays emitted from the light source unit 18 can be reflected almost regardless of the polarization state, and the amount of near-infrared rays with which the object B is irradiated can be improved.

In addition, it is preferable that the cholesteric liquid crystal layer has a pitch gradient structure in which a length of a helical pitch P changes continuously or intermittently in the thickness direction.

As is well known, in a case where light is incident from an oblique direction, the cholesteric liquid crystal layer causes a so-called blue shift (short wavelength shift) in which a wavelength of light to be selectively reflected varies to a short wavelength side. In contrast, by providing the cholesteric liquid crystal layer having a pitch gradient structure, the selective reflection wavelength range can be widened. Therefore, the cholesteric liquid crystal layer having a pitch gradient structure can suitably reflect the irradiated or scattered near-infrared rays regardless of the incidence direction of light into the liquid crystal diffraction element.

Hereinafter, an action of the blood flow measurement by the blood flow measurement device 10 will be described.

The blood flow measurement device 10 is mounted on and fixed to an object B, for example, a head (forehead), an arm, a leg, or the like. The blood flow measurement device 10 may be mounted on and fixed to the object B by a known method.

In a case of measuring the blood flow of the object B using the blood flow measurement device 10, near-infrared rays are emitted from the light source unit 18 in the irradiation unit 12.

As shown on the right side of FIG. 6, the near-infrared rays emitted from the light source unit 18 are transmitted through the irradiation unit guide plate 20, are incident on the incidence diffraction element 24, are diffracted by the incidence diffraction element 24 to be reflected, and are incident on the irradiation unit guide plate 20 at an angle at which total reflection can occur.

The near-infrared rays incident on the irradiation unit light guide plate 20 are guided in the irradiation unit light guide plate 20 by repeating total reflection, and are incident on the irradiation diffraction element 26.

The near-infrared rays incident on the irradiation diffraction element 26 are diffracted by the irradiation diffraction element 26 to be reflected, are emitted from the irradiation unit light guide plate 20, pass through an emission transmission window (not shown) provided in the case 36, and are emitted to the object B as indicated by an arrow Ni.

A part of the near-infrared rays with which the object B is irradiated is absorbed, and a part of the near-infrared rays is scattered, in a measurement target such as the vicinity of the cerebral cortex of the brain of the object B or the vicinity of a defect of the arm.

A part of the scattered light is emitted from the object B, is transmitted through the light receiving section light guide plate 30, and is incident on the light receiving diffraction element 34 as indicated by an arrow S.

The scattered light (near-infrared rays) incident on the light receiving diffraction element 34 is diffracted by the light receiving diffraction element 34 to be reflected, and a part of the light passes through an incidence transmission window not shown in the drawing, provided in the case 36, and is incident on the light receiving section light guide plate 30 at an angle at which total reflection can occur.

The scattered light incident on the light receiving section light guide plate 30 at an angle where total reflection can occur is guided in the light receiving section light guide plate 30 by repeating total reflection, and is incident on the emission diffraction element 32.

The near-infrared rays incident on the emission diffraction element 32 are diffracted by the irradiation diffraction element 26 to be reflected, are emitted from the light receiving section light guide plate 30, and are incident on the light receiving element 28 to be measured.

A photometry result (output signal) by the light receiving element 28 is, for example, amplified by an amplifier, and then converted into a digital signal by an AD converter to be sent to a control unit (not shown).

The control unit has, for example, a processor such as a central processing unit (CPU) or a digital signal processor (DSP) and a memory, and executes the processing of the signal and calculate the measurement result of the blood flow by a computer program, firmware, or the like that is developed on the memory in an executable manner. Furthermore, the control unit may be dedicated hardware such as a hardware circuit or a field programmable gate array (FPGA), which activates the light source unit 18 and the light receiving element 28, and executes a cooperation processing with each constituent.

For example, in the cerebral cortex of the brain, the blood flow rate changes in accordance with the activity state of the brain. As a result, the amount of oxygenated hemoglobin and the amount of non-oxygenated hemoglobin in the blood in each part of the cerebral cortex change depending on the blood flow rate. The absorption characteristics and/or the scattering characteristics of the near-infrared rays in the vicinity of the cerebral cortex change due to a change in the amount of hemoglobin, a change in the amount of oxygen, and the like.

That is, the intensity of the scattered light received by the light receiving section 14 (light receiving element 28) changes in accordance with the change in the blood flow rate in the cerebral cortex.

In addition, the change in the intensity of the scattered light varies depending on the wavelength of the near-infrared rays to be irradiated.

For example, it is assumed that near-infrared rays α at a certain wavelength and near-infrared rays β at a wavelength shorter than the near-infrared rays α are incident with an intensity of 100. In this case, the intensity of the scattered light changes according to the activity state of the cerebral cortex, that is, the blood flow rate. For example, at a certain point in time, the intensity of scattered light of the near-infrared rays α is 20 and the intensity of scattered light of the near-infrared rays β is 20, but at a certain point in time thereafter, various changes occur, for example, the intensity of scattered light of the near-infrared rays α is 10 and the intensity of scattered light of the near-infrared rays β is 5.

Accordingly, for example, by continuously calculating a ratio between the scattered light of the near-infrared rays α and the scattered light of the near-infrared rays β, it is possible to detect a change in the amount of hemoglobin and a change in the amount of oxygen, that is, a change in the blood flow rate, and from these results, for example, the activity state of the brain can be known.

Here, as described above, in the blood flow measurement device 10 of the embodiment of the present invention, the irradiation unit 12 has the irradiation unit light guide plate 20, and the light receiving section 14 has the light receiving section light guide plate 30.

Therefore, in the blood flow measurement device 10 of the embodiment of the present invention, the incidence position of the near-infrared rays on the object B in the irradiation unit 12 and the disposition position of the light source unit 18 (light source) can be spaced from each other. In addition, in the blood flow measurement device 10 of the embodiment of the present invention, the incidence position of the scattered light and the disposition position of the light receiving element 28 in the light receiving section 14 can be spaced apart from each other. That is, in the blood flow measurement device 10 of the embodiment of the present invention, the light guide plate and the diffraction element can be present at the measurement site.

As one of the factors that cause the blood flow measurement device to be thick, the light source and the light receiving element are exemplified. In the blood flow measurement device in the related art, the light source and the light receiving element must be disposed at the measurement site, which serves as a factor that prevents the device from being made thinner.

In contrast, according to the present invention using the light guide plate, as described above, the incidence position of the near-infrared rays on the object and the light source, and the incidence position of the scattered light from the object and the light receiving element can be spaced from each other, and only the light guide plate and the diffraction element can be present at the measurement site. As an example, in a case of considering the blood flow in the cerebral cortex (in the vicinity of the cerebral cortex) in the forehead, only the light guide plate and the diffraction element are mounted on the forehead as the measurement site, and the light source and the light receiving element having a thickness can be disposed, for example, around the temple (squama temporalis).

In addition, by using the diffraction element to perform the incidence and the emission of the near-infrared rays into and from the light guide plate, an increase in the thickness of the blood flow measurement device can be prevented.

Therefore, according to the blood flow measurement device of the embodiment of the present invention, the device can be significantly made thinner at a measurement site, that is, the blood flow measurement device can be made thinner.

Further, by adjusting the length of the light guide plate, the distance between the incidence position of the near-infrared rays on the object B and the incidence position of the scattered light on the light guide plate from the object B can be adjusted, and information on the blood flow at different depths from a surface of the human body can thus be obtained.

The above-described liquid crystal diffraction element is a reflection-type liquid crystal diffraction element, as shown on the right side of FIG. 6, where the incidence diffraction element 24 of the irradiation unit 12 is shown as an example.

However, as described above, in the blood flow measurement device of the embodiment of the present invention, as conceptually shown on the left side of FIG. 6, where the incidence diffraction element 25 is shown as an example for causing near-infrared rays to be incident on the irradiation unit light guide plate 20, a transmission-type diffraction element can also be used. As shown in FIG. 6, in a case where the transmission-type diffraction element is used, the disposition position of the diffraction element is the opposite side of the reflection-type diffraction element in the light guide plate.

Furthermore, in the blood flow measurement device of the embodiment of the present invention, as described above, the reflection-type diffraction element and the transmission-type diffraction element may be used in a mixed manner.

FIG. 7 conceptually shows an example of the transmission-type liquid crystal diffraction element.

The liquid crystal diffraction element shown in FIG. 7 has a support 50, an alignment film 52, and an optically anisotropic layer 56 as a liquid crystal layer. The support 50 and the alignment film 52 are the same as those described above.

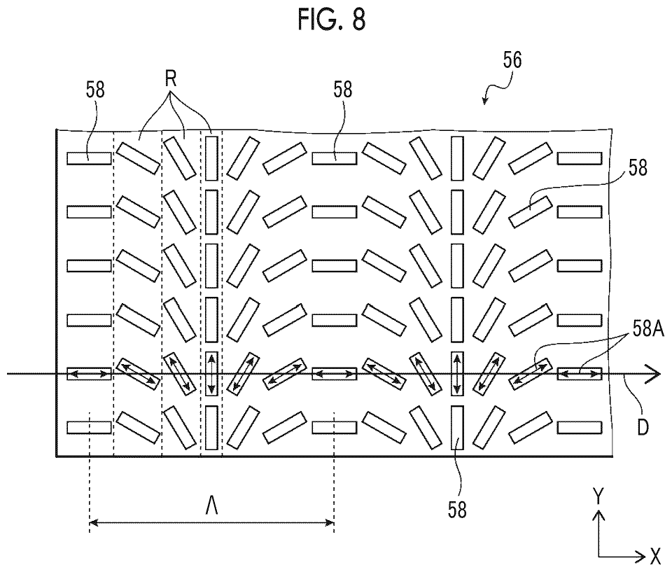

As conceptually shown in FIG. 8, the optically anisotropic layer 56 that is a liquid crystal layer constituting the transmission-type liquid crystal diffraction element also has a liquid crystal alignment pattern in which the optical axis 58A of the liquid crystal compound 58 continuously rotates along the arrangement axis D, as in the above-described cholesteric liquid crystal layer 54. Furthermore, FIG. 8 also shows only the liquid crystal compound 58 of a surface of the alignment film 52 as in FIG. 3.

In the liquid crystal diffraction element shown in FIG. 7, the liquid crystal compound 58 forming the optically anisotropic layer 56 is not helically twisted and does not rotate in the thickness direction, and the optical axis 58A is positioned at the same position in the plane direction. Such a liquid crystal layer can be formed by not adding a chiral agent to a liquid crystal composition during the formation of the above-described liquid crystal layer.

As described above, the optically anisotropic layer 56 has the liquid crystal alignment pattern in which the orientation of the optical axis 58A derived from the liquid crystal compound 58 changes while continuously rotating along the arrangement axis D direction in the plane, that is, in the X direction.

On the other hand, regarding the liquid crystal compound 58 forming the optically anisotropic layer 56, the liquid crystal compounds 58 having the same orientation of the optical axes 58A are arranged at regular intervals in the Y direction orthogonal to the X direction, that is, the Y direction orthogonal to the arrangement axis D as the one in-plane direction in which the optical axis 58A continuously rotates.

In other words, regarding the liquid crystal compound 58 forming the optically anisotropic layer 56, in the liquid crystal compounds 58 arranged in the Y direction, angles formed between the directions of the optical axes 58A and the arrangement axis D direction are the same.

In the liquid crystal compounds arranged in the Y direction in the optically anisotropic layer 56, the angles formed between the optical axes 58A and the X direction (the one direction in which the orientation of the optical axis of the liquid crystal compound 58 rotates) are the same. The regions in which the liquid crystal compounds 58 in which the angles formed between the optical axes 58A and the arrangement axis D are the same are disposed in the Y direction will be referred to as “regions R”.

In this case, it is preferable that the value of the in-plane retardation (Re) in each region R is a half wavelength, that is, λ/2. The in-plane retardation is calculated from the product of a difference Δn in refractive index generated by refractivity anisotropy of the region R and the thickness of the optically anisotropic layer. Here, the difference in refractive index generated by refractive index anisotropy of the region R in the optically anisotropic layer is defined by a difference between a refractive index of a direction of an in-plane slow axis of the region R and a refractive index of a direction orthogonal to the direction of the slow axis. That is, the difference in refractive index Δn due to the refractive index anisotropy of the regions R is the same as a difference between a refractive index of the liquid crystal compound 58 in the direction of the optical axis 58A and a refractive index of the liquid crystal compound 58 in the direction perpendicular to the optical axis 58A in the plane of the region R. That is, the difference Δn in refractive index is the same as the difference in refractive index of the liquid crystal compound 58.

In a case where circularly polarized light is incident on such the optically anisotropic layer 56, the light is refracted such that the direction of the circularly polarized light is converted.

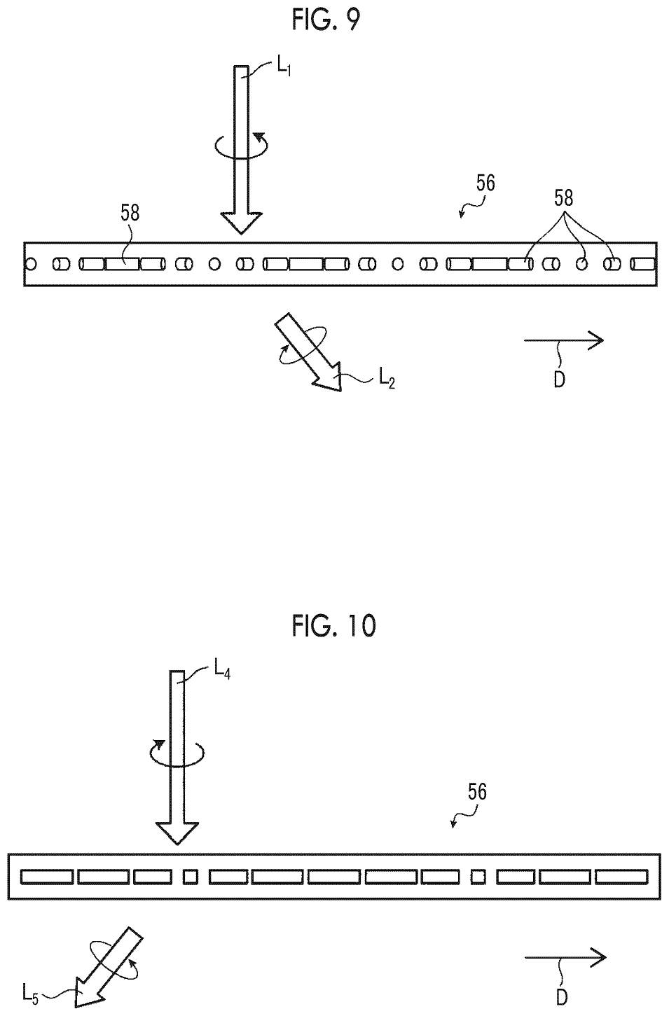

This action is conceptually shown in FIGS. 9 and 10. Furthermore, in the optically anisotropic layer 56, the value of the product of the difference in the refractive index of the liquid crystal compound and the thickness of the optically anisotropic layer is λ/2.

As shown in FIG. 9, in a case where a value of the product of the difference in refractive index of the liquid crystal compound of the optically anisotropic layer 56 and the thickness of the optically anisotropic layer 56 is λ/2 and incidence rays L₁ that is levorotatory circularly polarized light is incident on the optically anisotropic layer, the incidence rays L₁ passes through the optically anisotropic layer 56 to be imparted with a phase difference of 180° such that the transmission rays L₂ are converted into dextrorotatory circularly polarized light.

In addition, since the liquid crystal alignment pattern formed in the optically anisotropic layer 56 is a pattern that is periodic in the arrangement axis D direction, the transmission rays L₂ travel in a direction different from a traveling direction of the incidence rays L₁. In this way, the levorotatory circularly polarized incidence rays L₁ are converted into dextrorotatory circularly polarized transmission rays L₂, which are tilted by a predetermined angle in the arrangement axis D direction with respect to an incidence direction.

On the other hand, in a case where the value of the product of the difference in refractive index of the liquid crystal compound of the optically anisotropic layer 56 and the thickness of the optically anisotropic layer is λ/2, as shown in FIG. 10, as dextrorotatory circularly polarized incidence rays L₄ are incident on the optically anisotropic layer 56, the incidence rays L₄ pass through the optically anisotropic layer 56, thereby imparting a phase difference of 180°, and is converted into levorotatory circularly polarized transmission rays L₅.

In addition, since the liquid crystal alignment pattern formed in the optically anisotropic layer 56 is a pattern that is periodic in the arrangement axis D direction, the transmission rays L₅ travel in a direction different from a traveling direction of the incidence rays L₄. In this case, the transmission rays L₅ travel in a direction different from the transmission rays L₂, that is, in a direction opposite to the arrangement axis D direction with respect to the incidence direction. In this way, the incidence rays L₄ are converted into levorotatory circularly polarized transmission rays L₅, which are tilted by a predetermined angle in a direction opposite to the arrangement axis D direction with respect to the incidence direction.

As with the cholesteric liquid crystal layer 54, the optically anisotropic layer 56 can also adjust the angles of refraction of the transmission rays L₂ and L₅ by changing the single period Λ of the formed liquid crystal alignment pattern. Specifically, also in the optically anisotropic layer 56, as the single period Λ of the liquid crystal alignment pattern decreases, light components transmitted through the liquid crystal compounds 58 adjacent to each other more strongly interfere with each other. Therefore, the transmission ray components L₂ and L₅ can be more largely refracted.

For example, in a case where light is incident from the normal direction of the transmission-type liquid crystal diffraction element (optically anisotropic layer 56), an angle formed between the normal direction and a traveling direction of transmission rays increases as the single period Λ decreases.

Accordingly, the length of the single period Λ of the liquid crystal diffraction element may be appropriately set depending on the incidence angle into the light guide plate, the magnitude of diffraction of light to be emitted from the light guide plate, and the like.

In addition, with regard to the single period Λ of the irradiation diffraction element, as described above, the length of the single period Λ is preferably set so that an angle θ [°] formed by the irradiation direction of the near-infrared rays Ni from the irradiation diffraction element and the normal direction of the irradiation diffraction element satisfies the above-described “θ/d>0.5” in accordance with a distance d [mm] between the irradiation diffraction element and the light receiving diffraction element.

In addition, by reversing the rotation direction of the optical axis 58A of the liquid crystal compound 58, which rotates along the arrangement axis D direction, the refraction direction of transmission rays can be reversed. That is, in the example FIGS. 7 to 10, the rotation direction of the optical axis 58A toward the arrangement axis D direction is clockwise. By setting this rotation direction to be counterclockwise, the refraction direction of transmission rays can be reversed.

Furthermore, from the viewpoint of diffraction efficiency, also in a case where such a transmission-type liquid crystal diffraction element is used, it is preferable to use an optically anisotropic layer having a region in which the liquid crystal compound is helically twisted in the thickness direction and rotates (the twisted angle is less than 360°). As the diffraction efficiency is enhanced, the amount of the near-infrared rays incident on and guided in the light guide plate (the irradiation unit light guide plate 20 and the light receiving section light guide plate 30) can be increased.

Further, in the transmission-type liquid crystal diffraction element, by disposing two optically anisotropic layers in which the helical twisting directions in the thickness direction are opposite to each other by lamination, a higher diffraction efficiency can be obtained, which is more preferable.

In the blood flow measurement device using the transmission-type liquid crystal diffraction element (diffraction element), the near-infrared rays emitted from the light source unit are transmitted through the incidence diffraction element to be diffracted (refracted) such that the near-infrared rays are incident on the irradiation unit light guide plate at an angle at which total reflection can occur.

The near-infrared rays incident on the irradiation unit light guide plate are guided in the irradiation unit light guide plate by repeating total reflection, and are incident on the irradiation diffraction element.

The near-infrared rays incident on the irradiation diffraction element are diffracted by the irradiation diffraction element to be emitted from the irradiation unit light guide plate, and the object B is irradiated therewith.

As described above, a part of the near-infrared rays with which the object B is irradiated is absorbed and another part of the near-infrared rays is scattered, in a measurement target such as the vicinity of the cerebral cortex of the brain of the object B or the vicinity of a defect of the arm.

As described above, a part of the scattered light is emitted from the object B and transmitted through the light receiving diffraction element to be diffracted, and is incident on the light receiving section light guide plate at an angle at which total reflection can occur.

The scattered light (near-infrared rays) incident on the light receiving section light guide plate is guided in the irradiation unit light guide plate by repeating total reflection, and is incident on the emission diffraction element.

The scattered light incident on the emission diffraction element is transmitted through the emission diffraction element to be diffracted is emitted from the light receiving section light guide plate, is incident on the light receiving element, and is measured.

The above-described blood flow measurement device 10 has a configuration in which the irradiation unit 12 and the light receiving section 14 are housed in the case 36, but the blood flow measurement device of the embodiment of the present invention is not limited thereto.

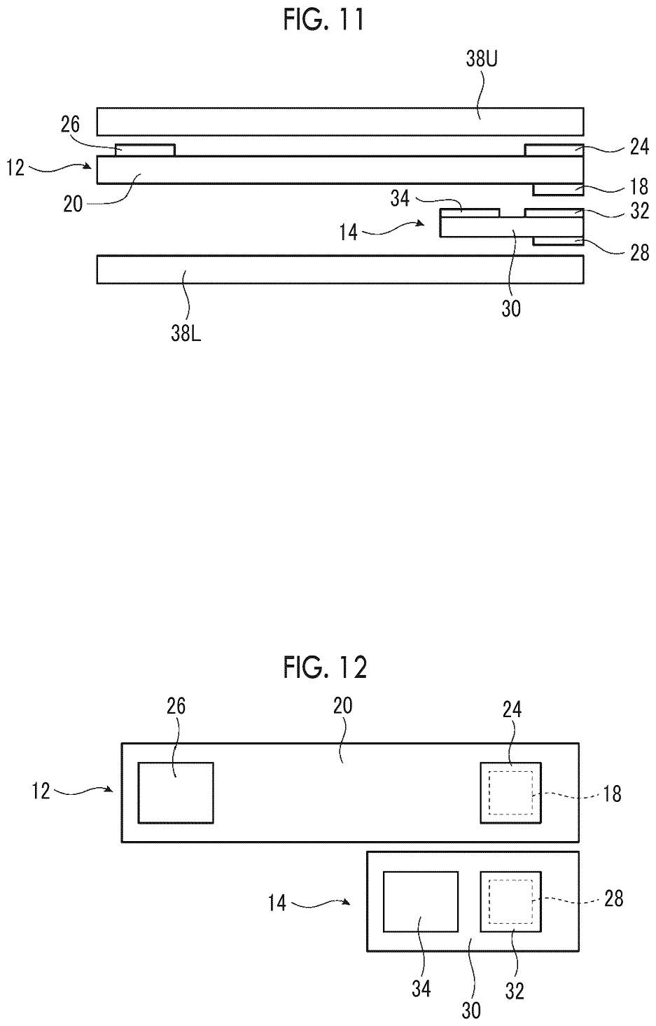

For example, a configuration in which an upper protective plate 38U and a lower protective plate 38L are provided to sandwich the laminated irradiation unit 12 and the laminated light receiving section 14 in the lamination direction, as conceptually shown in FIG. 11, can also be used.

This makes it possible to prevent the object B or the like from coming into contact with the irradiation unit 12 and the light receiving section 14, as in the above-described case 36.

Furthermore, as the upper protective plate 38U and the lower protective plate 38L, various known materials can be used as long as they do not come into contact with the irradiation unit 12 and the light receiving section 14 due to pressing or the like and have a strength to protect the irradiation unit 12 and the light receiving section 14.

The irradiation unit 12 and the light receiving section 14 are laminated in the above-described blood flow measurement device 10, but the blood flow measurement device of the embodiment of the present invention is not limited thereto.

That is, in the blood flow measurement device 10 of the embodiment of the present invention, the irradiation unit 12 and the light receiving section 14 may be arranged side by side in the plane direction as conceptually shown in FIG. 12. At this time as well, the liquid crystal alignment pattern is set so that the irradiation diffraction element 26 diffracts near-infrared rays toward the light receiving diffraction element 34.

Hereinbefore, the blood flow measurement device of the embodiment of the present invention has been described in detail, but the present invention is not limited to the above-described examples and various improvements and changes can be made without departing from the spirit of the present invention.

The present invention can be suitably used to measure a blood flow in blood vessels of the brain and the arm.

### EXPLANATION OF REFERENCES

10: blood flow measurement device
12: irradiation unit
14: light receiving section
18: light source unit
20: irradiation unit light guide plate
24: incidence diffraction element
26: irradiation diffraction element
28: light receiving element
30: light receiving section light guide plate
32: emission diffraction element
34: light receiving diffraction element
36: case
50: support
52: alignment film
54: cholesteric liquid crystal layer
56: optically anisotropic layer
58: liquid crystal compound
58A: optical axis
60: exposure device
62: laser
64: light source
65:22 plate
68: polarized beam splitter
70A, 70B: mirror
72A, 72B: λ/4 plate
B: object
R_{R}: dextrorotatory circularly polarized light of red light
M: laser light
MA, MB: rays
P_{O}: linearly polarized light
P_{R}: dextrorotatory circularly polarized light
P_{L}: levorotatory circularly polarized light
L₁, L₄: incidence rays
L₂, L₅: transmission rays
D: arrangement axis
Λ: single period (period of diffraction structure)
P: pitch

What is claimed is:

1. A blood flow measurement device comprising:
an irradiation unit that has a light source emitting near-infrared rays and irradiates an object with the near-infrared rays; and
a light receiving section that has a light receiving element for measuring scattered light that is irradiated from the irradiation unit and scattered by the object,
wherein in a case where a configuration in which the irradiation unit has an irradiation unit light guide plate that guides the near-infrared rays emitted from the light source and an irradiation diffraction element for emitting the near-infrared rays guided in the irradiation unit light guide plate from the irradiation unit light guide plate to irradiate the object with the near-infrared rays is defined as a first configuration, and
a configuration in which the light receiving section has a light receiving section light guide plate that guides the scattered light scattered by the object and a light receiving diffraction element for allowing the scattered light scattered by the object to be incident on the light receiving section light guide plate is defined as a second configuration,
the blood flow measurement device has at least one of the first configuration or the second configuration.

2. The blood flow measurement device according to claim 1,
wherein the blood flow measurement device has the first configuration and the second configuration.

3. The blood flow measurement device according to claim 1,
wherein at least one of the irradiation diffraction element or the light receiving diffraction element is a liquid crystal diffraction element.

4. The blood flow measurement device according to claim 3,
wherein the liquid crystal diffraction element includes a liquid crystal layer having a liquid crystal alignment pattern in which an orientation of an optical axis derived from the liquid crystal compound changes while continuously rotating along at least one in-plane direction.

5. The blood flow measurement device according to claim 4,
wherein a liquid crystal compound constituting the liquid crystal layer is helically twisted and aligned in a thickness direction.

6. The blood flow measurement device according to claim 4,
wherein the liquid crystal layer is a cholesteric liquid crystal layer.

7. The blood flow measurement device according to claim 5,
wherein the blood flow measurement device has two of the liquid crystal layers in which helical twisted directions of the liquid crystal compounds are different from each other.

8. The blood flow measurement device according to claim 6,
wherein the blood flow measurement device has two of the liquid crystal layers in which helical twisted directions of the liquid crystal compounds are different from each other.

9. The blood flow measurement device according to claim 1,
wherein the blood flow measurement device has at least one of the first configuration having an incidence diffraction element for allowing the near-infrared rays emitted from the light source to be incident on the irradiation unit light guide plate, or
the second configuration having an emission diffraction element for emitting the scattered light guided in the light receiving section light guide plate from the light receiving section light guide plate to be incident on the light receiving element.

10. The blood flow measurement device according to claim 2,
wherein the irradiation unit light guide plate and the light receiving section light guide plate are laminated.

11. The blood flow measurement device according to claim 2,
wherein the irradiation unit light guide plate and the light receiving section light guide plate are arranged in a plane direction.

12. The blood flow measurement device according to claim 1,
wherein at least one of the irradiation unit light guide plate or the light receiving section light guide plate has a first material and a second material having a refractive index higher than a refractive index of the first material, and
the second material is configured to be encompassed in the first material.

13. The blood flow measurement device according to claim 2,
wherein at least one of the irradiation diffraction element or the light receiving diffraction element is a liquid crystal diffraction element.

14. The blood flow measurement device according to claim 13,
wherein the liquid crystal diffraction element includes a liquid crystal layer having a liquid crystal alignment pattern in which an orientation of an optical axis derived from the liquid crystal compound changes while continuously rotating along at least one in-plane direction.

15. The blood flow measurement device according to claim 14,
wherein a liquid crystal compound constituting the liquid crystal layer is helically twisted and aligned in a thickness direction.

16. The blood flow measurement device according to claim 14,
wherein the liquid crystal layer is a cholesteric liquid crystal layer.

17. The blood flow measurement device according to claim 15,
wherein the blood flow measurement device has two of the liquid crystal layers in which helical twisted directions of the liquid crystal compounds are different from each other.

18. The blood flow measurement device according to claim 16,
wherein the blood flow measurement device has two of the liquid crystal layers in which helical twisted directions of the liquid crystal compounds are different from each other.

19. The blood flow measurement device according to claim 2,
wherein the blood flow measurement device has at least one of the first configuration having an incidence diffraction element for allowing the near-infrared rays emitted from the light source to be incident on the irradiation unit light guide plate, or
the second configuration having an emission diffraction element for emitting the scattered light guided in the light receiving section light guide plate from the light receiving section light guide plate to be incident on the light receiving element.

20. The blood flow measurement device according to claim 2,
wherein at least one of the irradiation unit light guide plate or the light receiving section light guide plate has a first material and a second material having a refractive index higher than a refractive index of the first material, and
the second material is configured to be encompassed in the first material.
